SEQUENCE LISTING (48 sequences)

SEQ ID NO: 1 - protein, 1118 aa, Clostridium histolyticum
MKKNILKILMDSYSKESKIQTVRRVTSVSLLAVYLTMNTSSLVLAKPIENTNDTSIKNVEKLRNAPNEENSKKVEDSKNDKVEHVKNIEEAKVEQVAPEVKSKSTLRSASIANTNSEKYDFEYLNGLSYTELTNLIKNIKWNQINGLFNYSTGSQKFFGDKNRVQAIINALQESGRTYTANDMKGIETFTEVLRAGFYLGYYNDGLSYLNDRNFQDKCIPAMIAIQKNPNFKLGTAVQDEVITSLGKLIGNASANAEVVNNCVPVLKQFRENLNQYAPDYVKGTAVNELIKGIEFDFSGAAYEKDVKTMPWYGKIDPFINELKALGLYGNITSATEWASDVGIYYLSKFGLYSTNRNDIVQSLEKAVDMYKYGKIAFVAMERITWDYDGIGSNGKKVDHDKFLDDAEKHYLPKTYTFDNGTFIIRAGDKVSEEKIKRLYWASREVKSQFHRVVGNDKALEVGNADDVLTMKIFNSPEEYKFNTNINGVSTDNGGLYIEPRGTFYTYERTPQQSIFSLEELFRHEYTHYLQARYLVDGLWGQGPFYEKNRLTWFDEGTAEFFAGSTRTSGVLPRKSILGYLAKDKVDHRYSLKKTLNSGYDDSDWMFYNYGFAVAHYLYEKDMPTFIKMNKAILNTDVKSYDEIIKKLSDDANKNTEYQNHIQELADKYQGAGIPLVSDDYLKDHGYKKASEVYSEISKAASLTNTSVTAEKSQYFNTFTLRGTYTGETSKGEFKDWDEMSKKLDGTLESLAKNSWSGYKTLTAYFTNYRVTSDNKVQYDVVFHGVLTDNADISNNKAPIAKVTGPSTGAVGRNIEFSGKDSKDEDGKIVSYDWDFGDGATSRGKNSVHAYKKAGTYNVTLKVTDDKGATATESFTIEIKNEDTTTPITKEMEPNDDIKEANGPIVEGVTVKGDLNGSDDADTFYFDVKEDGDVTIELPYSGSSNFTWLVYKEGDDQNHIASGIDKNNSKVGTFKSTKGRHYVFIYKHDSASNISYSLNIKGLGNEKLKEKENNDSSDKATVIPNFNTTMQGSLLGDDSRDYYSFEVKEEGEVNIELDKKDEFGVTWTLHPESNINDRITYGQVDGNKVSNKVKLRPGKYYLLVYKYSGSGNYELRVNK

SEQ ID NO: 2 - protein, 1021 aa, Clostridium histolyticum
MKRKCLSKRLMLAITMATIFTVNSTLPIYAAVDKNNATAAVQNESKRYTVSYLKTLNYYDLVDLLVKTEIENLPDLFQYSSDAKEFYGNKTRMSFIMDEIGRRAPQYTEIDHKGIPTLVEVVRAGFYLGFHNKELNEINKRSFKERVIPSILAIQKNPNFKLGTEVQDKIVSATGLLAGNETAPPEVVNNFTPILQDCIKNIDRYALDDLKSKALFNVLAAPTYDITEYLRATKEKPENTPWYGKIDGFINELKKLALYGKINDNNSWIIDNGIYHIAPLGKLHSNNKIGIETLTEVMKVYPYLSMQHLQSADQIKRHYDSKDAEGNKIPLDKFKKEGKEKYCPKTYTFDDGKVIIKAGARVEEEKVKRLYWASKEVNSQFFRVYGIDKPLEEGNPDDILTMVIYNSPEEYKLNSVLYGYDTNNGGMYIEPEGTFFTYEREAQESTYTLEELFRHEYTHYLQGRYAVPGQWGRTKLYDNDRLTWYEEGGAELFAGSTRTSGILPRKSIVSNIHNTTRNNRYKLSDTVHSKYGASFEFYNYACMFMDYMYNKDMGILNKLNDLAKNNDVDGYDNYIRDLSSNYALNDKYQDHMQERIDNYENLTVPFVADDYLVRHAYKNPNEIYSEISEVAKLKDAKSEVKKSQYFSTFTLRGSYTGGASKGKLEDQKAMNKFIDDSLKKLDTYSWSGYKTLTAYFTNYKVDSSNRVTYDVVFHGYLPNEGDSKNSLPYGKINGTYKGTEKEKIKFSSEGSFDPDGKIVSYEWDFGDGNKSNEENPEHSYDKVGTYTVKLKVTDDKGESSVSTTTAEIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 3 - DNA, 934 bp, Pichia pastoris
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 4 - DNA, 4906 bp, Pichia pastoris
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggaga

SEQ ID NO: 5 - DNA, 2646 bp, Pichia pastoris
cctgatgactgactcactgataataaaaatacggcttcagaatttctcaagactacactcactgtccgacttcaagtaatgacatttcccttgctacctgcatacgcaagtgttgcagagtttgataattccttgagtttggtaggaaaagccgtgtttccctatgctgctgaccagctgcacaacctgatcaagttcactcaatcgactgagcttcaagttaatgtgcaagttgagtcatccgttacagaggaccaatttgaggagctgatcgacaacttgctcaagttgtacaataatggtatcaatgaagtgattttggacctagatttggcagaaagagttgtccaaaggatcccaggcgctagggttatctataggaccctggttgataaagttgcatccttgcccgctaatgctagtatcgctgtgcctttttcttctccactgggcgatttgaaaagtttcactaatggcggtagtagaactgtttatgctttttctgagaccgcaaagttggtagatgtgacttccactgttgcttctggtataatccccattattgatgctcggcaattgactactgaatacgaactttctgaagatgtcaaaaagttccctgtcagtgaaattttgttggcgtctttgactactgaccgccccgatggtctattcactactttggtggctgactcttctaattactcgttgggcctggtgtactcgtccaaaaagtctattccggaggctataaggacacaaactggagtctaccaatctcgtcgtcacggtttgtggtataaaggtgctacatctggagcaactcaaaagttgctgggtatcgaattggattgtgatggagactgcttgaaatttgtggttgaacaaacaggtgttggtttctgtcacttggaacgcacttcctgttttggccaatcaaagggtcttagagccatggaagccaccttgtgggatcgtaagagcaatgctccagaaggttcttataccaaacggttatttgacgacgaagttttgttgaacgctaaaattagggaggaagctgatgaacttgcagaagctaaatccaaggaagatatagcctgggaatgtgctgacttattttattttgcattagttagatgtgccaagtacggtgtgacgttggacgaggtggagagaaacctggatatgaagtccctaaaggtcactagaaggaaaggagatgccaagccaggatacaccaaggaacaacctaaagaagaatccaaacctaaagaagtcccttctgaaggtcgtattgaattgtgcaaaattgacgtttctaaggcctcctcacaagaaattgaagatgcccttcgtcgtcctatccagaaaacggaacagattatggaattagtcaaaccaattgtcgacaatgttcgtcaaaatggtgacaaagcccttttagaactaactgccaagtttgatggagtcgctttgaagacacctgtgttagaagctcctttcccagaggaacttatgcaattgccagataacgttaagagagccattgatctctctatagataacgtcaggaaattccatgaagctcaactaacggagacgttgcaagttgagacttgccctggtgtagtctgctctcgttttgcaagacctattgagaaagttggcctctatattcctggtggaaccgcaattctgccttccacttccctgatgctgggtgttcctgccaaagttgctggtcgcaaagaaattgtttttgcatctccacctaagaaggatggtacccttaccccagaagtcatctacgttgcccacaaggttggtgctaagtgtatcgtgctagcaggaggcgcccaggcagtagctgctatggcttacggaacagaaactgttcctaagtgtgacaaaatatttggtccaggaaaccagttcgttactgctgccaagatgatggttcaaaatgacacatcagccctgtgtagtattgacatgcctgctgggccttctgaagttctagttattgctgataaatacgctgatccagatttcgttgcctcagaccttctgtctcaagctgaacatggtattgattcccaggtgattctgttggctgtcgatatgacagacaaggagcttgccagaattgaagatgctgttcacaaccaagctgtgcagttgccaagggttgaaattgtacgcaagtgtattgcacactctacaaccctatcggttgcaacctacgagcaggctttggaaatgtccaatcagtacgctcctgaacacttgatcctgcaaatcgagaatgcttcttcttatgttgatcaagtacaacacgctggatctgtgtttgttggtgcctactctccagagagttgtggagattactcctccggtaccaaccacactttgccaacgtacggatatgcccgtcaatacagcggagttaacactgcaaccttccagaagttcatcacttcacaagacgtaactcctgagggactgaaacatattggccaagcagtgatggatctggctgctgttgaaggtctagatgctcaccgcaatgctgttaaggttcgtatggagaaactgggacttatttaaattatttagagattttaacttacatttagattcgat

SEQ ID NO: 6 - DNA, 352 bp, Saccharomyces cerevisiae
atgagatttccttcaatttttactgcagttttattcgcagcatcctccgattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagaatgagatttccttcaatttttactgcagttttattcgcagcatcctccgatgagatttccttcaatttttactgcagttttattcgcagcatcctccg

SEQ ID NO: 7 - DNA, 30 bp, Artificial Sequence
  Multi Cloning Site
aagcttggatccagatcttctagagaattc

SEQ ID NO: 8 - DNA, 30 bp, Artificial Sequence
  pUC-pAOX
ataaggatccaacatccaaagacgaaaggt

SEQ ID NO: 9 - DNA, 30 bp, Artificial Sequence
  pUCpAOX
tataggatcccgtttcgaataattagttgt

SEQ ID NO: 10 - DNA, 30 bp, Artificial Sequence
  pUC-PaoxTaox
ataatctagatcaagaggatgtcagaatgc

SEQ ID NO: 11 - DNA, 30 bp, Artificial Sequence
  pUC-PaoxTaox
tatatctagatctcctcctccggttgcttt

SEQ ID NO: 12 - DNA, 30 bp, Artificial Sequence
  pUC-PaoxTaox
ataagaattccctgatgactgactcactga

SEQ ID NO: 13 - DNA, 30 bp, Artificial Sequence
  pUC-PaoxTaox
tatagaattcatcgaatctaaatgtaagtt

SEQ ID NO: 14 - DNA, 29 bp, Artificial Sequence
  BglII_MF
taatagatctatgaagaagaacatcttga

SEQ ID NO: 15 - DNA, 29 bp, Artificial Sequence
  ColG_BglII
taaagatctttatttatttactctcaact

SEQ ID NO: 16 - DNA, 33 bp, Artificial Sequence
  ColH_BglII
aataaagatctttatctaccaacagaaccttcg

SEQ ID NO: 17 - DNA, 33 bp, Artificial Sequence
  ColG_N149A_Fw
attaacggtctttttgcttactctactggttcc

SEQ ID NO: 18 - DNA, 33 bp, Artificial Sequence
  ColG_N149A_Rv
ggaaccagtagagtaagcaaaaagaccgttaat

SEQ ID NO: 19 - DNA, 33 bp, Artificial Sequence
  ColG_N251A_Fw
ggaaagttgattggtgctgcttccgccaatgcc

SEQ ID NO: 20 - DNA, 33 bp, Artificial Sequence
  ColG_N251A_Rv
ggcattggcggaagcagcaccaatcaactttcc

SEQ ID NO: 21 - DNA, 33 bp, Artificial Sequence
  ColG_N330A_Fw
ttgggtttgtacggtgctattacttctgctact

SEQ ID NO: 22 - DNA, 33 bp, Artificial Sequence
  ColG_N330A_Rv
agtagcagaagtaatagcaccgtacaaacccaa

SEQ ID NO: 23 - DNA, 33 bp, Artificial Sequence
  ColG_N419A_Fw
acctatacttttgatgctggaacctttattatc

SEQ ID NO: 24 - DNA, 33 bp, Artificial Sequence
  ColG_N419A_Rv
gataataaaggttccagcatcaaaagtataggt

SEQ ID NO: 25 - DNA, 33 bp, Artificial Sequence
  ColG_N704A_Fw
gccgcctctttgactgctacttctgttaccgct

SEQ ID NO: 26 - DNA, 33 bp, Artificial Sequence
  ColG_N704A_Rv
agcggtaacagaagtagcagtcaaagaggcggc

SEQ ID NO: 27 - DNA, 33 bp, Artificial Sequence
  ColG_N857A_Fw
aaggccggaacctacgctgtcactttgaaggtc

SEQ ID NO: 28 - DNA, 33 bp, Artificial Sequence
  ColG_N857A_Rv
gaccttcaaagtgacagcgtaggttccggcctt

SEQ ID NO: 29 - DNA, 33 bp, Artificial Sequence
  ColG_N915A_Fw
gtcaagggtgatcttgctggatccgacgacgcc

SEQ ID NO: 30 - DNA, 33 bp, Artificial Sequence
  ColG_N915A_Rv
ggcgtcgtcggatccagcaagatcacccttgac

SEQ ID NO: 31 - DNA, 33 bp, Artificial Sequence
  ColG_N944A_Fw
tactctggatcttccgcttttacttggcttgtc

SEQ ID NO: 32 - DNA, 33 bp, Artificial Sequence
  ColG_N944A_Rv
gacaagccaagtaaaagcggaagatccagagta

SEQ ID NO: 33 - DNA, 33 bp, Artificial Sequence
  ColG_N966A_Fw
tctggaattgataaggctaattctaaggttggt

SEQ ID NO: 34 - DNA, 33 bp, Artificial Sequence
  ColG_N966A_Rv
accaaccttagaattagccttatcaattccaga

SEQ ID NO: 35 - DNA, 33 bp, Artificial Sequence
  ColG_N992A_Fw
cacgactctgcttctgctatctcctactctttg

SEQ ID NO: 36 - DNA, 33 bp, Artificial Sequence
  ColG_N992A_Rv
caaagagtaggagatagcagaagcagagtcgtg

SEQ ID NO: 37 - DNA, 33 bp, Artificial Sequence
  ColG_S1015A_Fw
aaggaaaacaatgacgcctctgataaagccacc

SEQ ID NO: 38 - DNA, 33 bp, Artificial Sequence
  ColG_S1015A_Rv
ggtggctttatcagaggcgtcattgttttcctt

SEQ ID NO: 39 - DNA, 33 bp, Artificial Sequence
  ColH_N1026A_Fw
gtcatccctaatttcgctaccaccatgcaggga

SEQ ID NO: 40 - DNA, 33 bp, Artificial Sequence
  ColG_N1026A_Rv
tccctgcatggtggtagcgaaattagggatgac

SEQ ID NO: 41 - DNA, 25 bp, Artificial Sequence
  ColH_N89A_Fw
ttacggtgctaagactagaatgtct

SEQ ID NO: 42 - DNA, 30 bp, Artificial Sequence
  ColH_N89A_Rv
cattctagtcttagcaccgtaaaattcctt

SEQ ID NO: 43 - DNA, 26 bp, Artificial Sequence
  ColH_N180A_Fw
ttgttggctggtgctgagactgctcc

SEQ ID NO: 44 - DNA, 27 bp, Artificial Sequence
  ColH_N180A_Rv
agcagtctcagcaccagccaacaaacc

SEQ ID NO: 45 - DNA, 28 bp, Artificial Sequence
  ColH_N514A_Fw
taacatccatgctactactagaaacaac

SEQ ID NO: 46 - DNA, 26 bp, Artificial Sequence
  ColH_N514A_Rv
gtttctagtagtagcatggatgttag

SEQ ID NO: 47 - DNA, 30 bp, Artificial Sequence
  ColH_N601A_Fw
gataactacgaagctttgactgttcctttt

SEQ ID NO: 48 - DNA, 29 bp, Artificial Sequence
  ColH_N601A_Rv
ggaacagtcaaagcttcgtagttatcgat

(19) United States
(12) Patent Application Publication
(10) Pub. No.: US 2021/0009978 A1
(43) Pub. Date: Jan. 14, 2021

(54) POLYPEPTIDE HAVING COLLAGENASE ACTIVITY AND METHOD FOR PRODUCING THE SAME

(71) Applicant: KANEKA CORPORATION, Osaka (JP)

(72) Inventor: Masahiro Aratake, Hyogo (JP)

(73) Assignee: KANEKA CORPORATION, Osaka (JP)

(21) Appl. No.: 17/032,866

(22) Filed: Sep. 25, 2020

Related U.S. Application Data
(63) Continuation of application No. PCT/JP2019/004513, filed on Feb. 7, 2019.

(30) Foreign Application Priority Data
Mar. 26, 2018 (JP) 2018-058845

(51) Int. Cl. C12N 9/52; C12N 1/16

(52) U.S. Cl. CPC C12N 9/52; C12N 2510/02; C12N 1/16; C12Y 304/24003

(57) ABSTRACT

One or more embodiments of the present invention provide a novel polypeptide that enables production of a highly uniform polypeptide having collagenase activity. The polypeptide of the one or more embodiments of the present invention is a mutant polypeptide comprising amino acid substitutions in collagenase G or collagenase H. The mutant polypeptide is not modified with an N-linked sugar chain when it is obtained via secretory production in an expression system using a yeast host.

### TECHNICAL FIELD

One or more embodiments of the present invention relate to a polypeptide having collagenase activity, a vector comprising nucleotide sequences encoding the polypeptide and a signal peptide, and a yeast comprising nucleotide sequences encoding the polypeptide and a signal peptide.

### BACKGROUND

Collagenase G or H derived from Clostridium histolyticum is capable of specifically degrading a collagen having a triple helix structure. Thus, it is widely used as a cell dispersion material in the field of cytology. When producing collagenase G or H with the use of Clostridium histolyticum, collagenases G and H are simultaneously obtained by secretory production, and by-products are generated disadvantageously (Non-Patent Document 1). In order to obtain highly uniform collagenase G or H, accordingly, it was necessary to separate collagenases G and H from each other. In the case of recombinant production of collagenase G or H using E. coli hosts, degradation products of collagenase G or H were generated disadvantageously. In order to obtain highly uniform collagenase G or H, accordingly, it was necessary to remove the degradation products via affinity purification involving the use of tag sequences (Patent Document 1).
Patent Document 1: JP Patent No. 5,698,536
Non-Patent Document 1: Matsushita, O. et al., Journal of Bacteriology, 1999, 181, pp. 923-933

### SUMMARY

One or more embodiments of the present invention provide a novel polypeptide that enables production of a highly uniform polypeptide having collagenase activity.

In order to establish a technique of mass-producing uniform collagenase G or H derived from Clostridium histolyticum, a polypeptide was allowed to express in an expression system using a yeast host. The present inventors found that expressing the polypeptide of collagenase G or H in a yeast host would not produce degradation products as observed in other expression systems (Non-Patent Document 1; Patent Document 1).

When a polypeptide is expressed in a yeast host cell, it is known to be modified with N-linked sugar chains. It is known that the N-linked sugar chain is not uniform because of the sugar chain structure. In the experiments described herein, in addition, both wild-type collagenases G and H derived from Clostridium histolyticum were found to be obtained by secretory production in the form of polypeptides comprising N-linked sugar chains added thereto. In one or more embodiments of the present invention, a technique of producing a collagenase without the N-linked sugar chain by identifying the site of N-glycosylated modification and introducing an amino acid substitution for avoiding the modification had been studied. This has led to the completion of one or more embodiments of the present invention.

(1) A polypeptide comprising an amino acid sequence as defined in (a1) or (a2) and satisfying the condition (b), wherein the amino acid sequence is either (c1) or (c2):
(a1) an amino acid sequence having 85% or higher sequence identity to the amino acid sequence set forth in SEQ ID NO: 1 or 2, or
(a2) an amino acid sequence derived from the amino acid sequence set forth in SEQ ID NO: 1 or 2 by substitution, deletion, and/or addition of one or a plurality of amino acid residues;
(b) the polypeptide has collagenase activity; and
(c1) an amino acid sequence as defined in (a1) or (a2) in which all the amino acid residues corresponding to amino acids 149, 251, 330, 419, 704, 857, 915, 944, 966, 992, 1013, and 1026 in the amino acid sequence set forth in SEQ ID NO: 1 are resistant to N-glycosylated modification, or
(c2) an amino acid sequence as defined in (a1) or (a2) in which all the amino acid residues corresponding to amino acids 89, 180, 514, and 601 in the amino acid sequence set forth in SEQ ID NO: 2 are resistant to N-glycosylated modification.
(2) The polypeptide according to (1), wherein the amino acid sequence is (d1) or (d2) below:
(d1) an amino acid sequence derived from the amino acid sequence set forth in SEQ ID NO: 1 by amino acid substitutions comprising:

one or more amino acid substitutions at positions 149, 150, and 151, selected from a group consisting of substitution with an amino acid other than asparagine at position 149, substitution with proline at position 150, and substitution with an amino acid other than serine or threonine at position 151:

one or more amino acid substitutions at positions 251, 252, and 253, selected from a group consisting of substitution with an amino acid other than asparagine at position 251, substitution with proline at position 252, and substitution with an amino acid other than serine or threonine at position 253;

one or more amino acid substitutions at positions 330, 331, and 332, selected from a group consisting of substitution with an amino acid other than asparagine at position 330, substitution with proline at position 331, and substitution with an amino acid other than serine or threonine at position 332;

one or more amino acid substitutions at positions 419, 420, and 421, selected from a group consisting of substitution with an amino acid other than asparagine at position 419, substitution with proline at position 420, and substitution with an amino acid other than serine or threonine at position 421:

one or more amino acid substitutions at positions 704, 705, and 706, selected from a group consisting of substitution with an amino acid other than asparagine at position 704, substitution with proline at position 705, and substitution with an amino acid other than serine or threonine at position 706:

one or more amino acid substitutions at positions 857, 858, and 859, selected from a group consisting of substitution with an amino acid other than asparagine at position 857, substitution with proline at position 858, and substitution with an amino acid other than serine or threonine at position 859:

one or more amino acid substitutions at positions 915, 916, and 917, selected from a group consisting of substitution with an amino acid other than asparagine at position 915, substitution with proline at position 916, and substitution with an amino acid other than serine or threonine at position 917;

one or more amino acid substitutions at positions 944, 945, and 946, selected from a group consisting of substitution with an amino acid other than asparagine at position 944, substitution with proline at position 945, and substitution with an amino acid other than serine or threonine at position 946:

one or more amino acid substitutions at positions 966, 967, and 968, selected from a group consisting of substitution with an amino acid other than asparagine at position 966, substitution with proline at position 967, and substitution with an amino acid other than serine or threonine at position 968:

one or more amino acid substitutions at positions 992, 993, and 994, selected from a group consisting of substitution with an amino acid other than asparagine at position 992, substitution with proline at position 993, and substitution with an amino acid other than serine or threonine at position 994;

one or more amino acid substitutions at positions 1013, 1014, and 1015, selected from a group consisting of substitution with an amino acid other than asparagine at position 1013, substitution with proline at position 1014, and substitution with an amino acid other than serine or threonine at position 1015; and

one or more amino acid substitutions at positions 1026, 1027, and 1028, selected from a group consisting of substitution with an amino acid other than asparagine at position 1026, substitution with proline at position 1027, and substitution with an amino acid other than serine or threonine at position 1028; or

(d2) an amino acid sequence derived from the amino acid sequence set forth in SEQ ID NO: 2 by amino acid substitutions comprising:

one or more amino acid substitutions at positions 89, 90, and 91, selected from a group consisting of substitution with an amino acid other than asparagine at position 89, substitution with proline at position 90, and substitution with an amino acid other than serine or threonine at position 91;

one or more amino acid substitutions at positions 180, 181, and 182, selected from a group consisting of substitution with an amino acid other than asparagine at position 180, substitution with proline at position 181, and substitution with an amino acid other than serine or threonine at position 182;

one or more amino acid substitutions acids at positions 514, 515, and 516, selected from a group consisting of substitution with an amino acid other than asparagine at position 514, substitution with proline at position 515, and substitution with an amino acid other than serine or threonine at position 516; and one or more amino acid substitutions at positions 601, 602, and 603, selected from a group consisting of substitution with an amino acid other than asparagine at position 601, substitution with proline at position 602, and substitution with an amino acid other than serine or threonine at position 603.

(3) A vector comprising a polynucleotide comprising nucleotide sequences encoding the polypeptide according to (1) or (2) and a signal peptide that enables secretion of the polypeptide from a yeast.
(4) A yeast comprising a polynucleotide comprising nucleotide sequences encoding the polypeptide according to (1) or (2) and a signal peptide that enables secretion of the polypeptide from a yeast.

The present description encompasses the contents disclosed in JP Patent Application No. 2018-058845, to which the present application claims the priority.

According to one or more embodiments of the present invention, a highly uniform polypeptide having collagenase activity can be produced without addition of the N-linked sugar chain.

### BRIEF DESCRIPTION OF THE DRAWINGS

FIG. 1 shows a result of an electrophoresis conducted in Comparative Example 4 for wild-type collagenase G in the yeast culture supernatant (Lane 1) and a polypeptide resulting from cleavage of the N-linked sugar chain from the wild-type collagenase G (Lane 2).

FIG. 2 shows a result of an electrophoresis conducted in Example 5 for wild-type collagenase G in the yeast culture supernatant (Lane 1), a polypeptide resulting from cleavage of the N-linked sugar chain from the wild-type collagenase G (Lane 2), mutant collagenase G in the yeast culture supernatant (Lane 3), and a polypeptide resulting from removal of the N-linked sugar chain from the mutant collagenase G (Lane 4).

FIG. 3 shows a result of an electrophoresis conducted in Comparative Example 4 for wild-type collagenase H in the yeast culture supernatant (Lane 1) and a polypeptide resulting from cleavage of the N-linked sugar chain from the wild-type collagenase H (Lane 2).

FIG. 4 shows a result of an electrophoresis conducted in Example 5 for wild-type collagenase H in the yeast culture supernatant (Lane 1), a polypeptide resulting from cleavage of the N-linked sugar chain from the wild-type collagenase H (Lane 2), mutant collagenase H in the yeast culture supernatant (Lane 3), and a polypeptide resulting from removal of the N-linked sugar chain from the mutant collagenase H (Lane 4).

### DETAILED DESCRIPTION OF THE EMBODIMENTS

Hereafter, one or more embodiments of the present invention are described in detail.

### 1. Definition of Terms

In the present disclosure, amino acids are denoted as follows:

A=Ala=alanine, C=Cys=cysteine, D=Asp=aspartic acid, E=Glu=glutamic acid, F=Phe=phenylalanine, G=Gly=glycine, H=His=histidine, I=Ile=isoleucine, K=Lys=lycine, L=Leu=leucine, M=Met=methionine, N=Asn=asparagine, P=Pro=proline, Q=Gln=glutamine, R=Arg=arginine, S=Ser=serine, T=Thr=threonine, V=Val=valine, W=Trp=tryptophane, Y=Tyr=tyrosine

In the present disclosure, amino acids and proteins are denoted using abbreviations adopted by the IUPAC-IUB Commission on Biochemical Nomenclature (CBN) indicated below. In the amino acid sequence of the protein, the N terminus is indicated at the left end, and the C terminus is indicated at the right end, unless otherwise specified. The common nomenclature indicated below is employed for ease of reference. For example, substitution is indicated in the form of “original amino acid; position; substituted amino acid,” and substitution of tyrosine at position 64 with aspartic acid is indicated as “Y64D.” Multiple mutations are indicated with hyphenation. For example, “S41A-Y64D” indicates substitution of serine at position 41 with alanine and that of tyrosine at position 64 with aspartic acid.

In the present disclosure, nucleotide sequence and amino acid sequence identity can be determined using a method or sequence analysis software well known to a person skilled in the art. For example, the blastn program or blastp program of the BLAST algorithm or the fasta program of the FASTA algorithm can be used. In the present disclosure, the “sequence identity” between the target nucleotide sequence to be evaluated and the nucleotide sequence X is determined by aligning the nucleotide sequence X and the target nucleotide sequence to be evaluated, introducing a gap according to need, adjusting the degree of nucleotide consistency at the maximal level, and indicating a frequency of the identical nucleotides appearing at the identical sites in the nucleotide sequence including a gap portion in terms of percentage (%). When the nucleotide sequence of DNA is compared with the nucleotide sequence of RNA, T and U are regarded as identical to each other. In the present disclosure, “sequence identity” between the target amino acid sequence to be evaluated and the amino acid sequence X is determined by aligning the amino acid sequence X and the target amino acid sequence, introducing a gap according to need, adjusting the degree of amino acid consistency to the maximal level, and indicating a frequency of the identical amino acids appearing at the identical sites in the amino acid sequence including a gap portion in terms of percentage (%).

In the present disclosure, the term “polynucleotide” can be referred to as a “nucleic acid,” the term refers to DNA or RNA, and the term typically refers to DNA. In the present disclosure, a “polynucleotide” may be double-stranded with its complementary strand. When a “polynucleotide” is DNA, in particular, DNA comprising a given nucleotide sequence is preferably double-stranded with DNA comprising a complementary nucleotide sequence thereof.

In the present disclosure, a “polypeptide” refers to those in which two or more amino acids are peptide bonded, and includes, in addition to proteins, those having a short chain length called as peptides or oligopeptides.

In the present disclosure, a “nucleotide sequence encoding” a polypeptide refers to a nucleotide sequence of a polynucleotide that induces polypeptide production via transcription and translation. For example, the nucleotide sequence may be designed based on a codon table for a polypeptide consisting of an amino acid sequence.

The term “host” used herein refers to a cell that is transformed via introduction of a polynucleotide, and it is also referred to as a “host cell” or “transformant.”

In the present disclosure, the term “expression” refers to transcription or translation of a nucleotide sequence that generates a polypeptide. Such expression may be substantially constant regardless of external stimuli, growth conditions, and other factors, or expression may vary depending on such factors. A promoter that drives expression is not particularly limited, provided that such promoter drives expression of a nucleotide sequence encoding the polypeptide.

The term “polypeptide expression system” used herein refers to a host into which a polynucleotide comprising a nucleotide sequence encoding a polypeptide has been introduced and which can express and secrete the polypeptide.

In one or more embodiments of the present invention, a host organism species is preferably a yeast. A yeast may not be capable of assimilating methanol, and examples thereof include those of Saccharomyces, Schizosaccharomyces, Kluyveromyces, and Yarrowia. A yeast may be capable of assimilating methanol. A yeast capable of assimilating methanol, which is a methanol-utilizing yeast, is more preferable. In general, a methanol-utilizing yeast is defined as a yeast which can be cultured by utilizing methanol as the only carbon source. A yeast which originally was a methanol-utilizing yeast but has lost the methanol-utilizing ability due to an artificial modification or mutation is also encompassed by the methanol-utilizing yeast in one or more embodiments of the present invention.

Examples of methanol-utilizing yeast strains include yeasts belonging to the genus Pichia, the genus Ogataea, the genus Candida, the genus Torulopsis, and the genus Komagataella. Preferred examples of the genus Pichia includes Pichia methanolica. Preferred examples of the genus Ogataea include Ogataea angusta, Ogataea polymorpha, Ogataea parapolymorpha, and Ogataea minuta. Preferred examples of the genus Candida include Candida boidinii. Preferred examples of the genus Komagataella include Komagataella pastoris and Komagataella phaffii.

Among the methanol-utilizing yeast strains mentioned above, yeast strains belonging to the genus Pichia, the genus Komagataella, or the genus Ogataea are particularly preferable.

As the yeast belonging to the genus Komagataella, yeasts belonging to Komagataella pastoris and Komagataella phaffii are preferable. Komagataella pastoris and Komagataella phaffii both have another name as Pichia pastoris.

Specific examples of strains that can be used as hosts include Komagataella pastoris ATCC76273 (Y-11430, CBS7435) and Komagataella pastoris X-33. These strains are available from, for example, American Type Culture Collection or Thermo Fisher Scientific.

Yeast strains of Ogataea are preferably Ogataea angusta, Ogataea polymorpha, and Ogataea parapolymorpha. These three strains are related to each other and are also referred to as “Hansenula polymorpha” or “Pichia angusta.”

Specific examples of strains that can be used include Ogataea angusta NCYC495 (ATCC14754), Ogataea polymorpha 8V (ATCC34438), and Ogataea parapolymorpha DL-1 (ATCC26012). These strains are available from, for example. American Type Culture Collection.

In one or more embodiments of the present invention, strains derived from yeast strains such as those of the genus Pichia, the genus Komagataella, and the genus Ogataea can be used as hosts. An example of a histidine auxotrophic strain is the Komagataella pastoris GS115 strain (available from Thermo Fisher Scientific), and examples of leucine auxotrophic strains include BY4329 derived from NCYC495, BY5242 derived from 8V, and BY5243 derived from DL-1 (these strains can be provided by the National BioResource Project). In one or more embodiments of the present invention, strains derived from such strains can also be used.

The term “secretory production” refers to production of a polypeptide by a host cell. Specifically, a host cell containing a polynucleotide comprising a nucleotide sequence encoding a polypeptide of interest expresses the polypeptide and secretes it extracellularly.

When a polypeptide is “highly uniform” in one or more embodiments of the present invention, there is no collagenase degradation product at a site indicating a lower molecular weight than a deduced molecular weight of Clostridium histolyticum-derived collagenase G or H. Such polypeptide has a sugar chain structure that is not uniform, it has no N-linked sugar chain added thereto, and it has collagenase activity.

### 2. The Polypeptide According to One or More Embodiments of the Present Invention

When a polypeptide comprising a conserved sequence Asn-X1-X2 (wherein X1 represents an amino acid residue other than proline; and X2 represents serine or threonine) is expressed into an amino acid sequence in a yeast and secreted extracellularly, the N-linked sugar chain is added to Asn in the conserved sequence. It should be noted that the presence of the conserved sequence does not always allow the yeast to add the N-linked sugar chain to the Asn. Because of the influence of, for example, the three-dimensional structure in the vicinity of the conserved sequence in the polypeptide, the N-linked sugar chain may not be added to the Asn. In the amino acid sequence of wild-type collagenase G derived from Clostridium histolyticum set forth in SEQ ID NO: 1, amino acids 38 to 40 (Asn-Thr-Ser), amino acids 52 to 54 (Asn-Asp-Thr), amino acids 149 to 151 (Asn-Tyr-Ser), amino acids 251 to 253 (Asn-Ala-Ser), amino acids 330 to 332 (Asn-Ile-Thr), amino acids 419 to 421 (Asn-Gly-Thr), amino acids 704 to 706 (Asn-Thr-Ser), amino acids 857 to 859 (Asn-Val-Thr), amino acids 915 to 917 (Asn-Gly-Ser), amino acids 944 to 946 (Asn-Phe-Thr), amino acids 966 to 968 (Asn-Asn-Ser), amino acids 992 to 994 (Asn-Ile-Ser), amino acids 1013 to 1015 (Asn-Asp-Ser), and amino acids 1026 to 1028 (Asn-Thr-Thr) correspond to the conserved sequence described above. The present inventors found that, in the conserved sequence described above, the N-linked sugar chain would be added to Asn-Tyr-Ser at positions 149 to 151 (N149), Asn-Ala-Ser at positions 251 to 253 (N251), Asn-Ile-Thr at positions 330 to 332 (N330). Asn-Gly-Thr at positions 419 to 421 (N419), Asn-Thr-Ser at positions 704 to 706 (N704), Asn-Val-Thr at positions 857 to 859 (N857), Asn-Gly-Ser at positions 915 to 917 (N915), Asn-Phe-Thr at positions 944 to 946 (N944), Asn-Asn-Ser at positions 966 to 968 (N966), Asn-Ile-Ser at positions 992 to 994 (N992), Asn-Asp-Ser at positions 1013 to 1015 (N1013), and Asn-Thr-Thr at positions 1026 to 1028 (N1026) when wild-type collagenase G derived from Clostridium histolyticum is produced via secretory production using an expression system using a yeast host. In the amino acid sequence of wild-type collagenase H derived from Clostridium histolyticum set forth in SEQ ID NO: 2, Asn-Ser-Thr at positions 23 to 25, Asn-Ala-Thr at positions 36 to 38, Asn-Glu-Ser at positions 43 to 45. Asn-Lys-Thr at positions 89 to 91, Asn-Glu-Thr at positions 180 to 182, Asn-Phe-Thr at positions 190 to 192, Asn-Asn-Ser at positions 265 to 267, Asn-Thr-Thr at positions 514 to 516, Asn-Leu-Thr at positions 601 to 603, Asn-Gly-Thr at positions 733 to 735, Asn-Lys-Ser at positions 769 to 771. Asn-Asn-Ser at positions 912 to 914 Asn-Thr-Ser at positions 934 to 936, Asn-Leu-Ser at positions 984 to 986, and Asn-Gly-Ser at positions 1005 to 1007 correspond to the conserved sequence described above. The present inventors found that, in the conserved sequence described above, the N-linked sugar chain would be added to Asn-Lys-Thr at positions 89 to 91 (N89), Asn-Glu-Thr at positions 180 to 182 (N180), Asn-Thr-Thr at positions 514 to 516 (N514), and Asn-Leu-Thr at positions 601 to 603 (N601) when wild-type collagenase H derived from Clostridium histolyticum is produced via secretory production using an expression system using a yeast host. When a wild-type collagenase derived from Clostridium histolyticum comprising the amino acid sequence set forth in SEQ ID NO: 1 or 2 was expressed in an expression system using a Clostridium histolyticum or E. coli host according to a conventional technique, in contrast, the present inventors confirmed that the N-linked sugar chain would not be added, the degradation product was present, and the chemical structure was not uniform.

On the basis of the finding described above, the present inventors completed the polypeptide according to one or more embodiments of the present invention described in more detail below as a highly uniform polypeptide of a wild-type collagenase derived from Clostridium histolyticum using a polypeptide expression system using a yeast host.

One or more embodiments of the present invention relate to a polypeptide comprising an amino acid sequence as defined in (a1) or (a2) below and satisfying the condition (b), wherein the amino acid sequence is either (c1) or (c2):

(a1) an amino acid sequence having 85% or higher sequence identity to the amino acid sequence set forth in SEQ ID NO: 1 or 2, or
(a2) an amino acid sequence derived from the amino acid sequence set forth in SEQ ID NO: 1 or 2 by substitution, deletion, and/or addition of one or a plurality of amino acid residues;
(b) the polypeptide has collagenase activity; and
(c1) an amino acid sequence in which all the amino acid residues corresponding to amino acids 149, 251, 330, 419, 704, 857, 915, 944, 966, 992, 1013, and 1026 in the amino acid sequence set forth in SEQ ID NO: 1 are resistant to N-glycosylated modification, or
(c2) an amino acid sequence in which all the amino acid residues corresponding to amino acids 89, 180, 514, and 601 in the amino acid sequence set forth in SEQ ID NO: 2 are resistant to N-glycosylated modification.

In the amino acid sequence as defined in (a1), the sequence identity may be 86% or higher, such as 87% or higher, 88% or higher, 89% or higher, 90% or higher, 91% or higher, 92% or higher, 93% or higher, 94% or higher, 95% or higher, 96% or higher, 97% or higher, 98% or higher, or 99% or higher. It should be noted that the amino acid sequence as defined in (a1) is a mutant sequence of the amino acid sequence set forth in SEQ ID NO: 1 or 2, and the sequence identity between the amino acid sequence as defined in (at) and the amino acid sequence set forth in SEQ ID NO: 1 or 2 is less than 100%.

In the amino acid sequence as defined in (a2), the term “one or a plurality of” refers to, for example, 1 to 40, such as 1 to 35, 1 to 30, 1 to 25, 1 to 20, 1 to 15, 1 to 11, 2 to 40, 2 to 35, 2 to 30, 2 to 25, 2 to 20, 2 to 15, 2 to 11, 3 to 40, 3 to 35, 3 to 30, 3 to 25, 3 to 20, 3 to 15, 3 to 11, 4 to 40, 4 to 35, 4 to 30, 4 to 25, 4 to 20, 4 to 15, or 4 to 12.

A polypeptide “having collagenase activity” as defined in (b) above has enzymatic activity of cleaving the N-terminal side of Gly in the amino acid sequence Pro-X-Gly-Pro-Y in the collagen molecule having a triple helix structure.

When the polypeptide is produced by secretory production with the use of the polypeptide expression system using a yeast host, amino acid residues corresponding to Asn in the conserved sequences are not modified with the N-linked sugar chain. Thus, a polypeptide without the N-linked sugar chain is produced by secretory production. One or more yeast strains, such as a Komagataella yeast, may be used as a host for the polypeptide production. An example of a Komagataella yeast is Komagataella pastoris (Pichia pastoris).

The amino acid sequence is preferably either (d1) or (d2) below. As a result of such amino acid substitutions, a polypeptide resistant to N-glycosylated modification can be obtained.

(d1) An amino acid sequence derived from the amino acid sequence set forth in SEQ ID NO: 1 by amino acid substitutions comprising:
one or more amino acid substitutions at positions 149, 150, and 151 (specifically, one or more amino acid substitutions selected from among substitution with an amino acid other than asparagine at position 149, substitution with proline at position 150, and substitution with an amino acid other than serine or threonine at position 151);

one or more amino acid substitutions at positions 251, 252, and 253 (specifically, one or more amino acid substitutions selected from among substitution with an amino acid other than asparagine at position 251, substitution with proline at position 252, and substitution with an amino acid other than serine or threonine at position 253);

one or more amino acid substitutions at positions 330, 331, and 332 (specifically, one or more amino acid substitutions selected from among substitution with an amino acid other than asparagine at position 330, substitution with proline at position 331, and substitution with an amino acid other than serine or threonine at position 332);

one or more amino acid substitutions at positions 419, 420, and 421 (specifically, one or more amino acid substitutions selected from among substitution with an amino acid other than asparagine at position 419, substitution with proline at position 420, and substitution with an amino acid other than serine or threonine at position 421);

one or more amino acid substitutions at positions 704, 705, and 706 (specifically, one or more amino acid substitutions selected from among substitution with an amino acid other than asparagine at position 704, substitution with proline at position 705, and substitution with an amino acid other than serine or threonine at position 706);

one or more amino acid substitutions at positions 857, 858, and 859 (specifically, one or more amino acid substitutions selected from among substitution with an amino acid other than asparagine at position 857, substitution with proline at position 858, and substitution with an amino acid other than serine or threonine at position 859);

one or more amino acid substitutions at positions 915, 916, and 917 (specifically, one or more amino acid substitutions selected from among substitution with an amino acid other than asparagine at position 915, substitution with proline at position 916, and substitution with an amino acid other than serine or threonine at position 917);

one or more amino acid substitutions at positions 944, 945, and 946 (specifically, one or more amino acid substitutions selected from among substitution with an amino acid other than asparagine at position 944, substitution with proline at position 945, and substitution with an amino acid other than serine or threonine at position 946);

one or more amino acid substitutions at positions 966, 967, and 968 (specifically, one or more amino acid substitutions selected from among substitution with an amino acid other than asparagine at position 966, substitution with proline at position 967, and substitution with an amino acid other than serine or threonine at position 968);

one or more amino acid substitutions at positions 992, 993, and 994 (specifically, one or more amino acid substitutions selected from among substitution with an amino acid other than asparagine at position 992, substitution with proline at position 993, and substitution with an amino acid other than serine or threonine at position 994);

one or more amino acid substitutions at positions 1013, 1014, and 1015 (specifically, one or more amino acid substitutions selected from among substitution with an amino acid other than asparagine at position 1013, substitution with proline at position 1014, and substitution with an amino acid other than serine or threonine at position 1015); and

one or more amino acid substitutions at positions 1026, 1027, and 1028 (specifically, one or more amino acid substitutions selected from among substitution with an amino acid other than asparagine at position 1026, substitution with proline at position 1027, and substitution with an amino acid other than serine or threonine at position 1028).

(d2) An amino acid sequence derived from the amino acid sequence set forth in SEQ ID NO: 2 by amino acid substitutions comprising:

one or more amino acid substitutions at positions 89, 90, and 91 (specifically, one or more amino acid substitutions selected from among substitution with an amino acid other than asparagine at position 89, substitution with proline at position 90, and substitution with an amino acid other than serine or threonine at position 91);

one or more amino acid substitutions at positions 180, 181, and 182 (specifically, one or more amino acid substitutions selected from among substitution with an amino acid other than asparagine at position 180, substitution with proline at position 181, and substitution with an amino acid other than serine or threonine at position 182);

one or more amino acid substitutions at positions 514, 515, and 516 (specifically, one or more amino acid substitutions selected from among substitution with an amino acid other than asparagine at position 514, substitution with proline at position 515, and substitution with an amino acid other than serine or threonine at position 516); and

one or more amino acid substitutions at positions 601, 602, and 603 (specifically, one or more amino acid substitutions selected from among substitution with an amino acid other than asparagine at position 601, substitution with proline at position 602, and substitution with an amino acid other than serine or threonine at position 603).

The polypeptide according to one or more embodiments of the present invention may be in the form of a fusion polypeptide comprising an additional polypeptide conjugated to either or both of the N terminal and C terminal sides. Examples of other polypeptides include, but are not limited to, a signal peptide and a tag peptide. Specific examples of signal peptides are as described below. Examples of tag peptides include a tag peptide comprising a plurality of (e.g., 6 to 10) histidine residues (a histidine tag) and FLAG tag peptide.

### 3. Vector

An aspect of one or more embodiments of the present invention relates to a vector comprising a polynucleotide comprising nucleotide sequences encoding the polypeptide according to one or more embodiments of the present invention and a signal peptide that enables secretion of the polypeptide from a yeast.

The vector according to one or more embodiments of the present invention is an artificially constructed nucleic acid molecule, and it generally comprises an exogenous nucleotide sequence in the nucleic acid molecule. The vector according to one or more embodiments of the present invention can be introduced into a yeast and used for yeast transformation.

A signal peptide that enables secretion of the polypeptide according to one or more embodiments of the present invention from a yeast contained in the vector according to one or more embodiments of the present invention is not particularly limited. An example thereof is a mating factor α (MFα) derived from Saccharomyces cerevisiae. In addition, the signal sequences of acidic phosphatase (PHO1) of Ogataea angusta, acidic phosphatase (PHO1) of Komagataella pastoris, invertase (SUC2) of Saccharomyces cerevisiae, PLB1 of Saccharomyces cerevisiae can be used as a signal peptide that enables secretion of the polypeptide according to one or more embodiments of the present invention from a yeast.

In the vector according to one or more embodiments of the present invention, a polynucleotide comprising a nucleotide sequence encoding the signal peptide may be provided at the 5′ terminus of a polynucleotide comprising a nucleotide sequence encoding the polypeptide according to one or more embodiments of the present invention. The vector according to one or more embodiments of the present invention may further comprise a nucleotide sequence encoding the tag peptide at either or both of the 5′ terminus and the 3′ terminus of the polynucleotide comprising a nucleotide sequence encoding the polypeptide according to one or more embodiments of the present invention.

Nucleotide sequences encoding the polypeptide according to one or more embodiments of the present invention and the signal peptide can be inserted into an expression cassette and included in the vector in that state. An “expression cassette” is an expression system that comprises a nucleotide sequence encoding the polypeptide according to one or more embodiments of the present invention and the signal peptide and is capable of providing the state to express it as a polypeptide. The “state to express” refers to a state in which the polypeptide-encoding nucleotide sequence comprised in the expression cassette is arranged under the control of the elements required for gene expression in such a way as to be expressed in a yeast host. Examples of the element required for gene expression include a promoter and a terminator.

The vector according to one or more embodiments of the present invention can be in the form of, for example, a cyclic vector, a linear vector, or an artificial chromosome.

The “promoter” herein refers to a nucleotide sequence region located upstream of the nucleotide sequence encoding the polypeptide according to one or more embodiments of the present invention and the signal peptide, wherein various transcription regulators involved in promoting or suppressing transcription, in addition to an RNA polymerase, binds to or work with the region to read the nucleotide sequence which is a template, whereby a complementary RNA is synthesized (transcribed).

For the promoter expressing a polypeptide, a promoter achieving the expression under the presence of a selected carbon source may be suitably used, and is not particularly limited.

In the expression cassette of the vector according to one or more embodiments of the present invention, a terminator is located downstream of the nucleotide sequences encoding the polypeptide according to one or more embodiments of the present invention and the signal peptide. An adequate terminator can be selected in accordance with a promoter and a yeast host used.

The vector according to one or more embodiments of the present invention can further comprise, for example, nucleotide sequences of a cloning site containing one or more restriction enzyme recognition sites, an overlap region for use of the In-fusion Cloning System (Clontech) or Gibson Assembly System (New England Biolabs), and a selection marker gene (e.g., an auxotrophic complementary gene or drug-resistance gene). The vector according to one or more embodiments of the present invention can further comprise an autonomous replication sequence (ARS), a centromeric DNA sequence, and a telomeric DNA sequence in accordance with a host.

### 4. Yeast

One or more embodiments of the present invention also relate to a yeast comprising a polynucleotide comprising nucleotide sequences encoding the polypeptide according to one or more embodiments of the present invention and a signal peptide that enables secretion of the polypeptide from a yeast. The yeast according to one or more embodiments of the present invention can comprise the polynucleotide as a part of the vector according to one or more embodiments of the present invention.

Specific yeast species that can be used as hosts are as exemplified above.

As a method of introducing the polynucleotide into a yeast; i.e., a method of transformation, a known method can be adequately employed. Examples thereof include, but are not particularly limited to, an electroporation method, a lithium acetate method, and a spheroplast method. As a method of Komagataella pastoris transformation, for example, a method of electroporation described in “High efficiency transformation by electroporation of Pichia pastoris pretreated with lithium acetate and dithiothreitol” (Biotechniques, 2004 January; 36 (1): 152-4) is generally employed.

### 5. Method for Producing the Polypeptide According to One or More Embodiments of the Present Invention

The method for producing the polypeptide according to one or more embodiments of the present invention comprises a step of culturing yeast. The target polypeptide according to one or more embodiments of the present invention may be collected from a culture of the yeast obtained in the step of culture described above. The term “culture” used herein refers to, in addition to a culture supernatant, a culture cell, a culture cell debris or other types of culture products. Since the yeast according to one or more embodiments of the present invention is capable of secretory production of the polypeptide according to one or more embodiments of the present invention extracellularly, a culture supernatant is particularly preferable as a culture. Specifically, a method for producing the polypeptide according to one or more embodiments of the present invention with the use of the yeast according to one or more embodiments of the present invention is preferably a method of culturing the yeast according to one or more embodiments of the present invention and accumulating the polypeptide according to one or more embodiments of the present invention in the culture supernatant. Conditions for culturing yeast are not particularly limited and may be appropriately selected depending on the cell. Any culture medium containing a nutrient source that can be utilized by the cell can be used.

A common medium adequately comprising such nutrients can be used, and examples of nutrients include: carbon sources, for example, lactose, such as glucose, sucrose, and maltose, organic acids, such as acetic acid, citric acid, and propionic acid, alcohols, such as methanol, ethanol, and glycerol, hydrocarbons, such as paraffin, oils, such as soybean oil and rapeseed oil, and a mixture of any thereof; nitrogen sources, such as ammonium sulfate, ammonium phosphate, urea, yeast extract, meat extract, peptone, and corn steep liquor; and other nutrients, such as inorganic salts and vitamins. The culture can be performed by either batch culture or continuous culture.

When a yeast strain of Pichias or Ogalaea is used according one or more embodiments of the present invention, one or more types of the carbon sources selected from among glucose, glycerol, and methanol may be used. Such carbon source may be present at the start of culture, or it may be added during culture.

Yeast culture can be performed under general conditions. For example, culture can be performed at a pH of 2.5 to 10.0 and a temperature of 10° C. to 48° C. under an aerobic atmosphere for 10 hours to 10 days.

The polypeptide according to one or more embodiments of the present invention can be collected from the culture by a known purification method. A plurality of known purification methods may be combined appropriately. For example, a culture mixture containing the yeast according to one or more embodiments of the present invention and a medium is first subjected to centrifugation or filtration, and yeast cells are then removed from a culture supernatant. The culture supernatant is subjected to one or more techniques selected from among, for example, salting out (e.g., ammonium sulfate precipitation or sodium phosphate precipitation), solvent precipitation (e.g., a method of protein fraction precipitation using acetone or ethanol), dialysis, gel filtration chromatography, ion-exchange chromatography, hydrophobic chromatography, affinity chromatography, reversed-phase chromatography, and ultrafiltration. Thus, the polypeptide according to one or more embodiments of the present invention is collected from the culture supernatant.

### EXAMPLES

Hereafter, one or more embodiments of the present invention are described in greater detail with reference to the examples. However, these examples are not intended to limit the scope of one or more embodiments of the present invention. Detailed manipulation methods and the like for recombinant DNA techniques used in Examples below are described in the following literature: Molecular Cloning 2nd Edition (Cold Spring Harbor Laboratory Press, 1989), Current Protocols in Molecular Biology (Green Publishing Associates and Wiley-Interscience).

In the following examples, plasmids used to transform yeasts were prepared by introducing the constructed vector into E. coli HST6CR competent cells (manufactured by Takara Bio Inc.) and culturing and growing the obtained transformants. Preparation of plasmid from the plasmid-carrying strain was performed using a QIAprep spin miniprep kit (QIAGEN).

The AOX1 promoter (SEQ ID NO: 3), the AOX1 terminator (SEQ ID NO: 4), and the HIS4 gene (SEQ ID NO: 5) used for vector construction were prepared by PCR using, as a template, a mixture of chromosome DNA of the Komagataella pastoris ATCC76273 strain (the nucleotide sequence thereof is described in the European Molecular Biology Laboratory (EMBL) ACCESSION No. FR839628 to FR839631).

Synthetic DNA of the wild-type collagenase gene of Clostridium histolyticum provided with a signal sequence of the a mating factor (the MF sequence) (SEQ ID NO: 6) used for vector construction was prepared in accordance with the disclosed sequence information (Uniprot numbers: Q9X721 (collagenase G); Q46085 (collagenase H)).

PCR was carried out with the use of, for example, Prime STAR Max DNA polymerase (Takara Bio Inc.) under the conditions described in the instructions. Chromosome DNA was prepared from the Komagataella pasoris ATCC76273 strain using, for example, Dr. GenTLE® (Takara Bio Inc.) under the conditions described in the instructions.

### Comparative Example 1: Construction of Vector for Wild-Type Collagenase Expression

A gene fragment comprising HindIII-BanHI-BglII-XbaI-EcoRImultiple cloning sites (SEQ ID NO: 7) was fully synthesized, and the gene fragment was inserted into between HindIII-EcoRI sites of pUC19 (Takara Bio Inc.) to construct pUC-1.

Also, a nucleic acid fragment comprising BamHI recognition sequences added to both terminuses of the AOX1 promoter was prepared by PCR with Primer 1 (SEQ ID NO: 8) and Primer 2 (SEQ ID NO: 9) using the chromosome DNA mixture as a template, the fragment was treated with BamHI, and the resultant was inserted into the BamHI site of pUC-1 to construct pUC-Paox.

Subsequently, a nucleic acid fragment comprising XbaI recognition sequences added to both terminuses of the AOX1 promoter was prepared by PCR with Primer 3 (SEQ ID NO: 10) and Primer 4 (SEQ ID NO: 11) using the chromosome DNA mixture as a template, the fragment was treated with XbaI, and the resultant was inserted into the XbaI site of pUC-Paox to construct pUC-PaoxTaox.

Subsequently, a nucleic acid fragment comprising EcoRI recognition sequences added to both terminuses of the HIS4 gene was prepared by PCR with Primer 5 (SEQ ID NO: 12) and Primer 6 (SEQ ID NO: 13) using the chromosome DNA mixture as a template, the fragment was treated with EcoRI, and the resultant was inserted into the EcoRI site of pUC-PaoxTaox to construct pUC-PaoxTaoxHIS4.

Subsequently, a nucleic acid fragment comprising BglII recognition sequences added to both terminuses of the collagenase G or H gene comprising the MF sequence thereto was prepared by PCR, using the synthetic DNA as a template, with Primer 7 (SEQ ID NO: 14) and Primer 8 (SEQ ID NO: 15) for collagenase G or with Primer 7 (SEQ ID NO: 14) and Primer 9 (SEQ ID NO: 16) for collagenase H, the fragment was treated with BglII, and the resultant was inserted into the BglII site of pUC-PaoxTaoxHIS4 to construct pUC-PaoxColGTaoxHIS4 (collagenase G) or pUC-PaoxColHTaoxHIS4 (collagenase H). pUC-PaoxColGTaoxHIS4 (collagenase G) or pUC-PaoxColHTaoxHIS4 (collagenase H) is designed so as to allow the wild-type collagenase G or H gene to undergo secretory expression under the control of the AOX1 promoter.

### Comparative Example 2: Acquisition of Transformed Yeast

With the use of the vector for wild-type collagenase expression constructed in Comparative Example 1, Komagataella pastoris was transformed in the manner described below.

The histidine auxotrophic strain derived from the Komagataella pastoris ATCC76273 strain was inoculated in 3 ml of YPD medium (1% yeast extract bacto (Difco), 2% polypeptone (Nihon Pharmaceutical Co., Ltd.), and 2% glucose), and shaking culture was performed at 30° C. overnight to obtain a preculture mixture. The resulting preculture mixture (500 μl) was inoculated in 50 ml of YPD medium, shaking culture was performed until OD600 reached 1 to 1.5, the cells were collected (3000×g, 10 minutes, 20° C.), and the cells were resuspended in 10 ml of 50 mM potassium phosphate buffer (pH 7.5) containing 250 μl of 1 M DTT (final concentration: 25 mM).

The suspension was incubated at 30° C. for 15 minutes, the cells were collected (3000×g, 10 minutes, 20° C.), and the cells were washed with 50 ml of pre-cooled STM buffer (270 mM sucrose, 10 mM Tris-HCl, 1 mM magnesium chloride, pH 7.5). The cells were collected from the wash mixture (3000×g, 10 minutes, 4° C.), washed again with 25 ml of STM buffer, and then collected (3000×g, 10 minutes, 4° C.). In the end, the cells were suspended in 250 μl of ice-cooled STM buffer, and the resulting suspension was designated as a competent cell suspension.

With the use of the vector for wild-type collagenase expression constructed in Comparative Example 1 (i.e., pUC-PaoxColGTaoxHIS4 or pUC-PaoxCotHTaoxHIS4), E. coli was transformed, the resulting transformant was cultured in 2 ml of ampicillin-containing LB medium (1.6% tryptone (Difco), 0.5% yeast extract bacto (Difco), and 1% sodium chloride (Difco)), and pUC-PaoxColGTaoxHIS4 or pUC-PaoxColHTaoxHIS4 was obtained from the cells using the QIAprep spin miniprep kit (QIAGEN). This plasmid was treated with SalI to prepare a linear vector cleaved at the SalI recognition sequence within the HIS4 gene.

The competent cell suspension (60 μl) was mixed with 1 μl of the linear pUC-PaoxColGTaoxHIS4 or pUC-PaoxColHTaoxHIS4 solution, the mixture was introduced into an electroporation cuvette (Disposable Cuvette electrodes, inter-electrode distance: 2 mm, BM Equipment Co., Ltd.), electroporation was performed at 7.5 kV/cm, 25 ρF, and 200Ω, the cells were suspended in 1 ml of YPD medium, and the suspension was allowed to stand at 30° C. for 1 hour. Thereafter, the cells were collected (3000×g, 5 minutes, 20° C.), suspended in 1 ml of YNB medium (0.67% Yeast Nitrogen Base without Amino Acids (Difco)), and then collected again (3000×g, 5 minutes, 20° C.). After the cells were resuspended in an adequate amount of YNB medium, the cell suspension was applied to a YNB selection agar plate (0.67% Yeast Nitrogen Base without Amino Acids (Difco), 2% agarose, and 2% glucose), cells grown via static culture at 30° C. for 3 days were selected, and a yeast strain expressing wild-type collagenase G or H was obtained.

### Comparative Example 3: Culture of Transformed Yeast

The yeast strain expressing wild-type collagenase obtained in Comparative Example 2 was inoculated in 3 ml of BMGMY medium (1% yeast extract bacto (Difco), 2% polypeptone (Nihon Pharmaceutical Co., Ltd.), 0.34% Yeast Nitrogen Base without Amino Acids and Ammonium Sulfate, 1% ammonium sulfate, 0.4 mg/l biotin, 100 mM potassium phosphate (pH 7.0), 1% glycerol, and 1% methanol), the resultant was subjected to shaking culture at 30° C. for 72 hours, and the culture supernatant was then collected via centrifugation (12,000 rpm, 5 minutes, 4° C.).

### Comparative Example 4: SDS-PAGE of Culture Supernatant

The culture supernatant obtained in Comparative Example 3 was analyzed via SDS-PAGE.

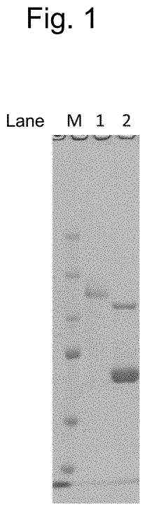

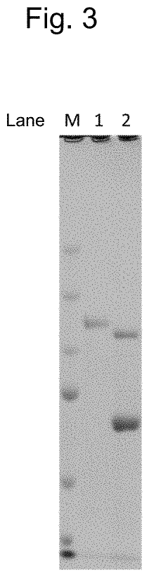

The culture supernatant (8 μl) was mixed with 8 μl of 2×sample buffer (0.25 M Tris-HCl (pH 6.8), 40% glycerol, 8% sodium dodecyl sulfate, and 0.02% bromophenol blue), and the resultant was treated at 95° C. for 8 minutes. The sample and the molecular weight marker (Precision Plus Protein™ Dual Color Standards, Bio-Rad) were subjected to SDS-PAGE electrophoresis using e-PAGEL gel (E-R7.5L, ATTO). Thereafter, the gel was rinsed for 15 minutes, stained for 30 minutes, and then decolored with water. As a result, a smear band was observed at a site indicating a molecular weight higher than the molecular weight deduced on the basis of the amino acid sequence (collagenase G: Lane 1, FIG. 1; collagenase H: Lane 1, FIG. 3). As a result of treatment of the culture supernatant with the N-linked sugar chain cleavage kit (Endo Hf, BioLabs), the smear band observed at a site indicating a higher molecular weight disappeared, and another band was observed at a position indicating the molecular weight deduced on the basis of the amino acid sequence (collagenase G: Lane 2, FIG. 1; collagenase H: Lane 2, FIG. 3). In the experiment in which wild-type collagenase G or H was obtained by secretory production with the aid of a Komagataella yeast, a degradation product observed at a site indicating a molecular weight lower than the deduced molecular weight upon expression in an E. coli host (Patent Document 1) was not observed (Lane 1, FIG. 1; Lane 1, FIG. 3). Since the band indicating collagenases G and H shifted toward a side indicating a lower molecular weight with the use of the N-linked sugar chain cleavage kit (Lane 2, FIG. 1; Lane 2, FIG. 3), it was confirmed that the Komagataella yeast produced wild-type collagenases G and H in the form modified with the N-linked sugar chain.

### Example 1: Construction of Vector for Mutant Collagenase Expression

With the use of the synthetic gene of the wild-type collagenase G or H gene comprising the MF sequence or a mutant gene prepared by PCR described below as a template, various mutant genes were prepared by PCR.

At the outset, PCR was performed using the wild-type collagenase G or H gene comprising the MF sequence or a mutant collagenase G or H gene comprising the MF sequence (any of Mutants 1 to 16) as a template and the primers in combination (1st PCR-1 and 1st PCR-2) as shown in Table 1, the resulting fragments were mixed, the resulting mixture was used as a template to perform PCR with the use of Primer 7 and Primer 8 for collagenase G or Primer 7 and Primer 9 for collagenase H, and a DNA fragment comprising BglII recognition sequences at both terminuses of any of various mutant collagenase genes comprising the MF sequences was prepared.

The DNA fragment containing the mutant collagenase gene prepared above was treated with BglII, the resultant was inserted into the BglII site of pUC-PaoxTaoxHIS4 prepared in Comparative Example 1, and vectors for expression of various mutant collagenase genes comprising the MF sequence were thus constructed.

TABLE 1

| | Template | Mutant | | | | | |
|---|---|---|---|---|---|---|---|
| Polypeptide | gene | gene | Site of mutation | 1st PCR-1 | | 1st PCR-2 | |
| Collagenase G | Wild-type | Mutant 1 | N149A | Primer 7 | Primer 11 | Primer 10 | Primer 8 |
| | | | | | SEQ ID NO: 18 | SEQ ID NO: 17 | |
| | Mutant 1 | Mutant 2 | N149A-N251A | Primer 7 | Primer 13 | Primer 12 | Primer 8 |
| | | | | | SEQ ID NO: 20 | SEQ ID NO: 19 | |
| | Mutant 2 | Mutant 3 | N149A-N251A-N330A | Primer 7 | Primer 15 | Primer 14 | Primer 8 |
| | | | | | SEQ ID NO: 22 | SEQ ID NO: 21 | |
| | Mutant 3 | Mutant 4 | N149A-N251A-N330A-N419A | Primer 7 | Primer 17 | Primer 16 | Primer 8 |
| | | | | | SEQ ID NO: 24 | SEQ ID NO: 23 | |
| | Mutant 4 | Mutant 5 | N149A-N251A-N330A-N419A-N704A | Primer 7 | Primer 19 | Primer 18 | Primer 8 |
| | | | | | SEQ ID NO: 26 | SEQ ID NO: 25 | |
| | Mutant 5 | Mutant 6 | N149A-N251A-N330A-N419A-N704A-N857A | Primer 7 | Primer 21 | Primer 20 | Primer 8 |
| | | | | | SEQ ID NO: 28 | SEQ ID NO: 27 | |
| | Mutant 6 | Mutant 7 | N149A-N251A-N330A-N419A-N704A-N857A- | Primer 7 | Primer 23 | Primer 22 | Primer 8 |
| | | | N915A | | SEQ ID NO: 30 | SEQ ID NO: 29 | |
| | Mutant 7 | Mutant 8 | N149A-N251A-N330A-N419A-N704A-N857A- | Primer 7 | Primer 25 | Primer 24 | Primer 8 |
| | | | N915A-N944A | | SEQ ID NO: 32 | SEQ ID NO: 31 | |
| | Mutant 8 | Mutant 9 | N149A-N251A-N330A-N419A-N704A-N857A- | Primer 7 | Primer 27 | Primer 26 | Primer 8 |
| | | | N915A-N944A-N966A | | SEQ ID NO: 34 | SEQ ID NO: 33 | |
| | Mutant 9 | Mutant 10 | N149A-N251A-N330A-N419A-N704A-N857A- | Primer 7 | Primer 29 | Primer 28 | Primer 8 |
| | | | N915A-N944A-N966A-N992A | | SEQ ID NO: 36 | SEQ ID NO: 35 | |
| | Mutant 10 | Mutant 11 | N149A-N251A-N330A-N419A-N704A-N857A- | Primer 7 | Primer 31 | Primer 30 | Primer 8 |
| | | | N915A-N944A-N966A-N992A-S1015A | | SEQ ID NO: 38 | SEQ ID NO: 37 | |
| | Mutant 11 | Mutant 12 | N149A-N251A-N330A-N419A-N704A-N857A- | Primer 7 | Primer 33 | Primer 32 | Primer 8 |
| | | | N915A-N944A-N966A-N992A-S1015A-N1026A | | SEQ ID NO: 40 | SEQ ID NO: 39 | |
| Collagenase H | Wild-type | Mutant 13 | N89A | Primer 7 | Primer 35 | Primer 34 | Primer 9 |
| | | | | | SEQ ID NO: 42 | SEQ ID NO: 41 | |
| | Mutant 13 | Mutant 14 | N89A-N180A | Primer 7 | Primer 37 | Primer 36 | Primer 9 |
| | | | | | SEQ ID NO: 44 | SEQ ID NO: 43 | |
| | Mutant 14 | Mutant 15 | N89A-N180A-N514A | Primer 7 | Primer 39 | Primer 38 | Primer 9 |
| | | | | | SEQ ID NO: 46 | SEQ ID NO: 45 | |
| | Mutant 15 | Mutant 16 | N89A-N180A-N514A-N601A | Primer 7 | Primer 41 | Primer 40 | Primer 9 |
| | | | | | SEQ ID NO: 48 | SEQ ID NO: 47 | |

### Example 2: Acquisition of Transformed Yeast

With the use of the vectors for mutant collagenase expression comprising the MF sequences constructed in Example 1, Komagataella pastoris was transformed in the same manner as described in Comparative Example 2.

E. coli strains were transformed using the vectors for mutant collagenase expression constructed in Example 1, the resulting transformants were cultured in 2 ml of ampicillin-containing LB medium, and a plasmid was obtained from the cells. The plasmid was treated with SalI and linearized.

Preparation of competent cells, transformation, and selection of transformants were performed in accordance with the methods described in Comparative Example 2.

### Example 3: Culture of Transformed Yeast

The mutant collagenase-expressing yeast strain obtained in Example 2 was cultured by the method described in Comparative Example 3, and the culture supernatant was collected.

### Example 4: Measurement of Collagenase Activity in Culture Supernatant

Collagenase activity of the mutant collagenase-expressing yeast strain obtained in Example 3 in the culture supernatant was measured in the manner described below.

To microdialyzers (Mini Dialysis Kit, 8 kDa cut-off, up to 250 μl, GE Healthcare), 200 μl each of the culture supernatant was added, and the resultant was stirred in a dialysis buffer (50 mM Tris-HCl (pH 7.6), 150 mM NaCl) at 4° C. overnight, followed by dialysis. The resulting solution was collected via centrifugation.

Incubation was performed using 200 μl of a reaction buffer (0.8 M Tris-HCl (pH 7.1), 0.2 M CaCl₂, and 1.23 mM 4-phenylazobenzyloxycarbonyl-Pro-Leu-Gly-Pro-D-Arg (Sigma-Aldrich)) at 37° C. 10 μl of the solution obtained in the dialysis was added thereto, and the resulting mixture was then subjected to incubation at 37° C. for 30 minutes. Thereafter, a 25 mM citric acid solution was added to terminate the reaction, and 2.5 ml of ethyl acetate was added. The resultant was subjected to inversion agitation for 15 seconds, and the organic layer (the upper layer) was transferred to another tube. Sodium sulfate (150 mg) was added thereto. The solution was analyzed using a spectrophotometer (U-2900, HITACHI) to determine the absorbance at 320 nm.

On the basis of the change in the absorbance at 320 nm 30 minutes after the initiation of the reaction, collagenase activity in the culture mixture was determined. A collagenase activity unit is an activity of improving the absorbance at 320 nm by 1.0 within a period of 1 minute in a reaction of 1 μmol of the reaction substrate (phenylazobenzyloxycarbonyl-Pro-Leu-Gly-Pro-D-Arg) at 37° C.

Collagenase activity was observed in the culture supernatants of various mutant collagenase-expressing strains.

TABLE 2

| | | | Collagenase |
|---|---|---|---|
| Poly- | | | activity |
| peptide | Mutant | Site of mutation | (U/ml) |
| Collage- | Wild-type | None | 0.0255 |
| nase G | Mutant 12 | N149A-N251A-N330A-N419A- | 0.0306 |
| | | N704A-N857A-N915A-N944A- | |
| | | N966A-N992A-S1015A-N1026A | |
| Collage- | Wildi-type | None | 3.68 |
| nase H | Mutant 16 | N89A-N180A-N514A-N601A | 4.03 |

### Example 5: SDS-PAGE of Culture Supernatant

The culture supernatant obtained in Example 3 was analyzed via SDS-PAGE. Analysis was performed in the same manner as in Comparative Example 4.

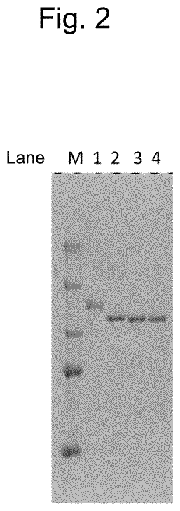

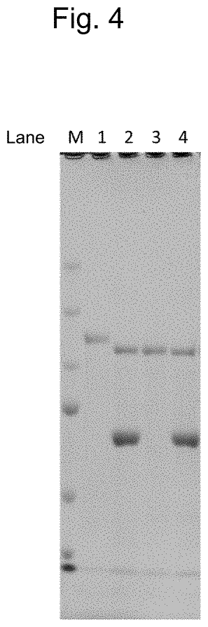

Unlike the wild-type collagenases (wild-type collagenase G: Lane 1. FIG. 2; wild-type collagenase H: Lane 1, FIG. 4), various mutant collagenases were found to show specific bands at positions corresponding to the deduced molecular weights (Lane 3, FIG. 2: Mutant 12 as a collagenase G mutant; Lane 3, FIG. 4: Mutant 16 as a collagenase H mutant; Lanes 2 and 4. FIG. 2: polypeptides resulting from cleavage of N-linked sugar chains from wild-type collagenase G and Mutant 12; Lanes 2 and 4, FIG. 4: polypeptides resulting from cleavage of N-linked sugar chains from wild-type collagenase H and Mutant 16; and Lanes 2, 3, and 4, FIG. 2 and Lanes 2, 3, and 4. FIG. 4: bands correspond to the deduced molecular weights) and found to be secreted without N-glycosylated modification. According to Patent Document 1, when the polypeptides were expressed in E. coli, a band indicating the degradation product having a lower molecular weight than the deduced molecular weight was observed. In contrast, however, no band indicating the degradation product was observed according to one or more embodiments of the present invention.

All publications, patents, and patent applications cited herein are incorporated herein by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A polypeptide comprising an amino acid sequence that is either (a1) or (a2):
(a1) an amino acid sequence having 85% or higher sequence identity to an amino acid sequence set forth in SEQ ID NO: 1 or 2,
(a2) an amino acid sequence derived from an amino acid sequence set forth in SEQ ID NO: 1 or 2 by substitution, deletion, and/or addition of 1 or a plurality of amino acid residues,
wherein the polypeptide has collagenase activity, and
wherein the amino acid sequence is either (c1) or (c2):
(c1) the amino acid sequence according to (a1) or (a2) wherein all an amino acid residues corresponding to amino acids 149, 251, 330, 419, 704, 857, 915, 944, 966, 992, 1013, and 1026 in the amino acid sequence set forth in SEQ ID NO: 1 are resistant to N-glycosylated modification;
(c2) the amino acid sequence according to (a1) or (a2) wherein all the amino acid residues corresponding to amino acids 89, 180, 514, and 601 in the amino acid sequence set forth in SEQ ID NO: 2 are resistant to N-glycosylated modification.

2. The polypeptide according to claim 1, wherein the amino acid sequence is either (d1) or (d2):
(d1) an amino acid sequence derived from the amino acid sequence set forth in SEQ ID NO: 1 by amino acid substitutions comprising:
one or more amino acid substitutions at positions 149, 150, and 151, selected from a group consisting of substitution with an amino acid other than asparagine at position 149, substitution with proline at position 150, and substitution with an amino acid other than serine or threonine at position 151;
one or more amino acid substitutions at positions 251, 252, and 253, selected from a group consisting of substitution with an amino acid other than asparagine at position 251, substitution with proline at position 252, and substitution with an amino acid other than serine or threonine at position 253;
one or more amino acid substitutions at positions 330, 331, and 332, selected from a group consisting of substitution with an amino acid other than asparagine at position 330, substitution with proline at position 331, and substitution with an amino acid other than serine or threonine at position 332;
one or more amino acid substitutions at positions 419, 420, and 421, selected from a group consisting of substitution with an amino acid other than asparagine at position 419, substitution with proline at position 420, and substitution with an amino acid other than serine or threonine at position 421;
one or more amino acid substitutions at positions 704, 705, and 706, selected from a group consisting of substitution with an amino acid other than asparagine at position 704, substitution with proline at position 705, and substitution with an amino acid other than serine or threonine at position 706;
one or more amino acid substitutions at positions 857, 858, and 859, selected from a group consisting of substitution with an amino acid other than asparagine at position 857, substitution with proline at position 858, and substitution with an amino acid other than serine or threonine at position 859;
one or more amino acid substitutions at positions 915, 916, and 917, selected from a group consisting of substitution with an amino acid other than asparagine at position 915, substitution with proline at position 916, and substitution with an amino acid other than serine or threonine at position 917;
one or more amino acid substitutions at positions 944, 945, and 946, selected from a group consisting of substitution with an amino acid other than asparagine at position 944, substitution with proline at position 945, and substitution with an amino acid other than serine or threonine at position 946;
one or more amino acid substitutions at positions 966, 967, and 968, selected from a group consisting of substitution with an amino acid other than asparagine at position 966, substitution with proline at position 967, and substitution with an amino acid other than serine or threonine at position 968;
one or more amino acid substitutions at positions 992, 993, and 994, selected from a group consisting of substitution with an amino acid other than asparagine at position 992, substitution with proline at position 993, and substitution with an amino acid other than serine or threonine at position 994;
one or more amino acid substitutions at positions 1013, 1014, and 1015, selected from a group consisting of substitution with an amino acid other than asparagine at position 1013, substitution with proline at position 1014, and substitution with an amino acid other than serine or threonine at position 1015; and
one or more amino acid substitutions at positions 1026, 1027, and 1028, selected from a group consisting of substitution with an amino acid other than asparagine at position 1026, substitution with proline at position 1027, and substitution with an amino acid other than serine or threonine at position 1028;
(d2) an amino acid sequence derived from the amino acid sequence set forth in SEQ ID NO: 2 by amino acid substitutions comprising:
one or more amino acid substitutions at positions 89, 90, and 91, selected from a group consisting of substitution with an amino acid other than asparagine at position 89, substitution with proline at position 90, and substitution with an amino acid other than serine or threonine at position 91;
one or more amino acid substitutions at positions 180, 181, and 182, selected from a group consisting of substitution with an amino acid other than asparagine at position 180, substitution with proline at position 181, and substitution with an amino acid other than serine or threonine at position 182;
one or more amino acid substitutions acids at positions 514, 515, and 516, selected from a group consisting of substitution with an amino acid other than asparagine at position 514, substitution with proline at position 515, and substitution with an amino acid other than serine or threonine at position 516; and
one or more amino acid substitutions at positions 601, 602, and 603, selected from a group consisting of substitution with an amino acid other than asparagine at position 601, substitution with proline at position 602, and substitution with an amino acid other than serine or threonine at position 603.

3. A vector comprising:
a polynucleotide comprising nucleotide sequences encoding the polypeptide according to claim 1; and
a signal peptide that enables secretion of the polypeptide from a yeast.

4. A yeast comprising:
a polynucleotide comprising nucleotide sequences encoding the polypeptide according to claim 1; and
a signal peptide that enables secretion of the polypeptide from the yeast.
